(19) United States
(12) Patent Application Publication
(10) Pub. No.: US 2024/0219049 A1
(43) Pub. Date: Jul. 4, 2024

(54) AIR STERILIZER AND AIR CONDITIONING APPARATUS USING THE SAME

(71) Applicant: Hitachi, Ltd., Chiyoda-ku, Tokyo (JP)

(72) Inventors: Hiroyuki TOYODA, Tokyo (JP); Hiroshi MAITA, Tokyo (JP); Shoichi TAGUCHI, Tokyo (JP); Daisuke AKIMARU, Tokyo (JP); Akinori KAWASAKI, Tokyo (JP)

(73) Assignee: Hitachi, Ltd., Chiyoda-ku, Tokyo (JP)

(21) Appl. No.: 17/920,086

(22) PCT Filed: Apr. 21, 2021
(86) PCT No.: PCT/JP2021/016169

§ 371 Date: Oct. 20, 2022

(51) Int. Cl. F24F 8/22; A61L 9/20

(52) U.S. Cl. CPC F24F 8/22; A61L 9/20

(57) ABSTRACT

Provided is an air sterilizer capable of efficiently irradiating dust, bacteria, viruses, and the like in circulating air with ultraviolet rays while preventing an increase in a blowing resistance, and an air conditioning apparatus using the air sterilizer.

An air sterilizer includes a frame of which air passes through an inside; an ultraviolet ray light source configured to emit ultraviolet light having a predetermined divergence angle; and a mirror surface configured to reflect the ultraviolet light and emit the ultraviolet light toward the air passing through the inside of the frame. A thickness of the frame in a direction in which the air passes is smaller than a diameter of a circle tangent to an inner circumference of the frame. When the ultraviolet light emitted from the ultraviolet ray light source is reflected by the mirror surface, the reflected light has a divergence angle or a convergence angle smaller than the divergence angle at least in a thickness direction of the frame.

### TECHNICAL FIELD

The present invention relates to an air sterilizer and an air conditioning apparatus using the same.

### BACKGROUND ART

In indoors such as houses, offices, factories, and commercial facilities, and in interiors of transportation means such as airplanes, railways, ships, and vehicles, most of floating dust, bacteria, viruses, and the like are naturally discharged to outsides by ventilation or the like, but some of the floating dust, bacteria, viruses, and the like remain in the indoors and in the interiors of the vehicles, and there is concern that the dust, the bacteria, the viruses and the like are diffused through an air circulation by an air conditioner. In recent years, due to an increasing interest in novel infectious diseases and the like that frequently occur, bacteria, viruses, and the like remain indoors and in interiors of vehicles are strongly required to be reduced more than before.

It is known that deep ultraviolet rays having a wavelength of 200 nm to 300 nm are effective in sterilizing bacteria and molds, inactivating viruses, and inactivating allergens such as pollen.

For example, PTL 1 discloses a technique of sterilizing (inactivating) bacteria, viruses, and the like in circulating air using ultraviolet rays.

### CITATION LIST

### Patent Literature

PTL 1: Japanese Patent No. 6587783 specification

### SUMMARY OF INVENTION

### Technical Problem

PTL 1 discloses that an ultraviolet ray light emitting diode (hereinafter abbreviated as an ultraviolet LED) is used as a light source of ultraviolet rays that inactivate bacteria, viruses, and the like. The ultraviolet LED has an advantage of having a relatively long life and being inexpensive, but also has a disadvantage that an intensity of an ultraviolet ray irradiated from the ultraviolet LED is generally low.

In order to efficiently inactivate bacteria, viruses, and the like in air, it is necessary to introduce air containing the bacteria, the viruses, and the like into a space having a high ultraviolet ray light density (inactivation space) and irradiate the bacteria, the viruses, and the like with ultraviolet rays having a sufficient intensity. In order to create such an inactivation space using the ultraviolet LED, it is necessary to mount a plurality of ultraviolet LEDs, to provide a reflection mirror for reflecting ultraviolet rays emitted from the ultraviolet LEDs to irradiate the bacteria, the viruses, and the like, and to ensure a long irradiation time of ultraviolet rays with respect to an air flow.

In order to secure a space for disposing a plurality of ultraviolet LEDs and reflection mirrors and to secure a long time for irradiating the bacteria, the viruses, and the like contained in air with ultraviolet rays, one idea is to secure the inactivation space as long as possible along a direction of the air flow.

Further, it is possible to provide a composite apparatus capable of realizing a plurality of functions by incorporating an ultraviolet ray irradiation device for inactivating bacteria, viruses, and the like into an air conditioning apparatus or an air purifier. In such a case, in order to reduce a size of the composite apparatus, a space in an internal duct of the air conditioning apparatus or the air purifier can be used as an inactivation space of the ultraviolet ray irradiation device.

However, in an internal duct of a general air conditioning apparatus or an air purifier, it is necessary to flow a large amount of air per unit time, and reduction of a blowing resistance is also an important problem. In a general air conditioning apparatus or the like, a large cross-sectional area of a cross section through which air flows is secured in order to reduce a blowing resistance while improving a heat exchange performance of a heat exchanger. Therefore, when inside of an internal duct of an air conditioning apparatus or the like is used as an inactivation space, extending a length of the duct in order to irradiate flowing air with a sufficient amount of ultraviolet light causes a problem that the blowing resistance is increased.

The invention is made in view of the problems of the related art, and an object of the invention is to provide an air sterilizer capable of efficiently irradiating dust, bacteria, viruses, and the like in circulating air with ultraviolet rays while preventing an increase in a blowing resistance, and an air conditioning apparatus using the air sterilizer.

### Solution to Problem

In order to achieve the above object, one typical air sterilizer according to the invention is achieved by including a frame of which air passes through an inside, an ultraviolet ray light source configured to emit ultraviolet light having a predetermined divergence angle, and a mirror surface configured to reflect the ultraviolet light and emit the ultraviolet light toward the air passing through the inside of the frame. A thickness of the frame in a direction in which the air passes is smaller than a diameter of a circle tangent to an inner circumference of the frame. When the ultraviolet light emitted from the ultraviolet ray light source is reflected by the mirror surface, the reflected light has a divergence angle or a convergence angle smaller than the divergence angle at least in a thickness direction of the frame.

### Advantageous Effects of Invention

According to the invention, it is possible to provide the air sterilizer capable of efficiently irradiating dust, bacteria, viruses, and the like in circulating air with ultraviolet rays while preventing the increase in the blowing resistance, and an air conditioning apparatus using the air sterilizer.

Problems to be solved, configurations, and effects other than those described above will be clarified by description of the following embodiments.

### BRIEF DESCRIPTION OF DRAWINGS

FIG. 1 is a perspective view of an air sterilizer according to a first embodiment of the invention.

FIG. 2 is a cross-sectional view in an axial direction of the air sterilizer according to the first embodiment of the invention.

FIG. 3 is a cross-sectional view in a direction orthogonal to an axis of the air sterilizer according to the first embodiment of the invention.

FIG. 4 is a perspective view of an air sterilizer according to a second embodiment of the invention.

FIG. 5 is a cross-sectional view in an axial direction of the air sterilizer according to the second embodiment of the invention.

FIG. 6 is a cross-sectional view in a direction orthogonal to an axis of the air sterilizer according to the second embodiment of the invention.

FIG. 7 is a diagram illustrating a mounting structure of an air sterilizer according to a third embodiment of the invention.

FIG. 8 is a cross-sectional view of a duct configuration using the air sterilizer according to the third embodiment of the invention.

FIG. 9 is a diagram illustrating a partial cut model of the duct configuration using the air sterilizer according to the third embodiment of the invention.

FIG. 10 is a diagram illustrating components of a fin tube heat exchanger used in the third embodiment of the invention.

FIG. 11 is a diagram illustrating a configuration of an air conditioning apparatus using an air sterilizer according to a fourth embodiment of the invention.

FIG. 12 is a mounting diagram of an air sterilizer in an air conditioning apparatus or a duct according to a fifth embodiment of the invention.

FIG. 13 is a perspective view of an air sterilizer according to a sixth embodiment of the invention.

FIG. 14 is a cross-sectional view in an axial direction of the air sterilizer according to the sixth embodiment of the invention.

FIG. 15 is a cross-sectional view in a direction orthogonal to an axis of the air sterilizer according to the sixth embodiment of the invention.

### DESCRIPTION OF EMBODIMENTS

Hereinafter, embodiments of the invention will be described with reference to the drawings.

### First Embodiment

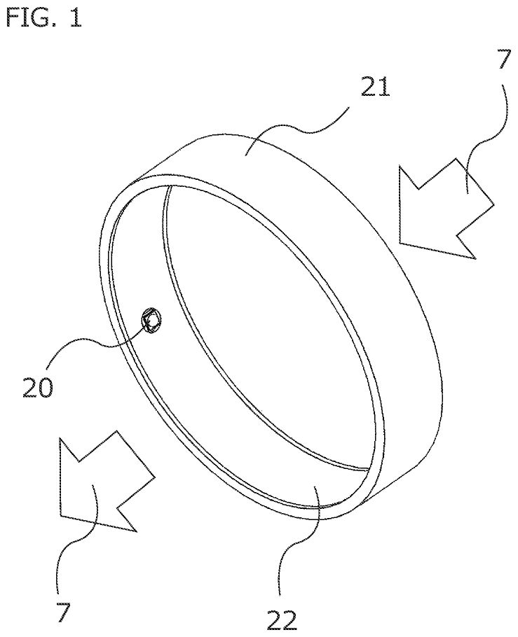

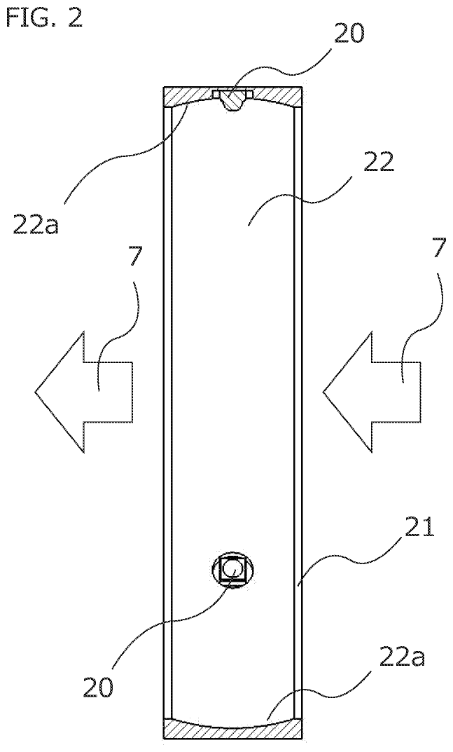

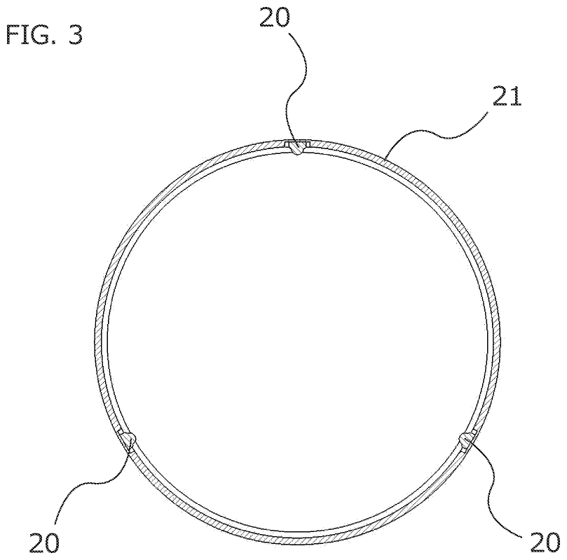

A first embodiment of the invention will be described with reference to FIGS. 1, 2, and 3. FIG. 1 is a perspective view of an air sterilizer according to the first embodiment. FIGS. 2 and 3 are views illustrating cross-sectional structures of the air sterilizer. However, the invention is not limited to these embodiments.

As illustrated in FIG. 1, in the air sterilizer of the present embodiment, a plurality of ultraviolet LEDs as ultraviolet ray light sources 20 are mounted inside a cylindrical frame 21 along a circumferential direction. The ultraviolet ray light source 20 is supplied with electric power from a power source (not illustrated), and is capable of emitting ultraviolet light from an emission surface that faces radially inward of the frame 21. An inner surface of the frame 21 is a mirror surface portion 22. The mirror surface portion 22 has a mirror surface 22a except for regions where the ultraviolet ray light sources 20 are disposed. The mirror surface 22a can reflect the ultraviolet light emitted from the ultraviolet ray light source 20 with a high reflectance. The mirror surface 22a has a function of repeatedly reflecting the ultraviolet light emitted from the ultraviolet ray light source 20 radially inward to keep the ultraviolet light inside the frame 21 of a cylindrical shape, thereby increasing an ultraviolet light density on a cylindrical inner surface.

Air flows in a direction indicated by an arrow of an air flow 7. That is, air flows in a thickness direction (that is, an axial direction) of the cylindrical frame 21.

As illustrated in FIG. 1, a diameter of a cylindrical inner circumferential surface of the frame 21 is larger than an axial thickness of the frame 21. This is because a duct through which air flows of an air conditioning apparatus or the like includes a structure such as a heat exchanger or a filter, and in order to avoid interference with such a structure, an air sterilizer mounted in an empty space in the duct preferably has a thin shape with respect to the air flow. Further, by reducing the axial thickness of the frame 21, an increase in a blowing resistance can be prevented.

Ultraviolet rays emitted from the ultraviolet ray light source 20 are particularly referred to as deep ultraviolet rays having a wavelength of about 200 nm to 300 nm. When bacteria, viruses and the like are irradiated with the ultraviolet rays, proteins, which are forming substances of the bacteria, the viruses and the like are destroyed. Therefore, inactivation of the bacteria, the viruses, and the like can be achieved by applying the ultraviolet rays having a wavelength of about 200 nm to 300 nm to the bacteria, the viruses, and the like.

Although the amount of energy required to inactivate bacteria, viruses, and the like varies depending on a wavelength of ultraviolet light, it is possible to inactivate bacteria, viruses, and the like by emitting a predetermined amount of the ultraviolet light having any wavelength between 200 nm and 300 nm. Currently, products that output light having a wavelength of 280 nm are available in the market as LEDs that emit deep ultraviolet rays, and in particular, in the present embodiment, it is assumed that ultraviolet LEDs having a wavelength of 280 nm or less are mounted.

In order to inactivate bacteria, viruses, and the like by irradiation of ultraviolet light having a constant energy, it is necessary to ensure the ultraviolet light density and irradiation time, but it is difficult to ensure the irradiation time in an irradiation space having no thickness in a flowing direction in flowing air, and thus it is important to increase the ultraviolet light density.

FIG. 2 is a cross-sectional view taken along a cross section parallel to a thickness direction of the cylindrical air sterilizer illustrated in FIG. 1. As illustrated in FIG. 2, a plurality of ultraviolet ray light sources 20 are provided so as to be embedded in a center in the thickness direction on an inner surface side of the frame 21. The inner surface of the frame 21 is the mirror surface portion 22 except for the ultraviolet ray light sources 20, and the mirror surface portion 22 has the mirror surface 22a having a curved surface shape which is a concave surface bulging outward when taken along the cross section illustrated in FIG. 2. The ultraviolet light emitted from the ultraviolet LED, which is the ultraviolet ray light source 20, is divergent light that spreads at a constant angle. This spread angle (referred to as a half-value angle) is preferably, for example, 40° to 60°.

The ultraviolet light emitted from the ultraviolet ray light source 20 travels while spreading outward in the thickness direction of the frame 21 at a predetermined divergence angle, and an optical axis of the ultraviolet light is orthogonal to an axis of the frame 21 and is reflected inward in the thickness direction (at least in the thickness direction of the frame 21, at a divergence angle (including a case where the divergence angle is 0°) or a convergence angle smaller than the divergence angle of the ultraviolet light emitted from the ultraviolet ray light source 20) by the facing mirror surface (concave surface) 22a, so that the amount of light leaking to an outside the frame 21 can be prevented, and the ultraviolet light density inside the frame 21 can be increased.

The shape of the mirror surface (concave surface) 22a may be an arc whose curvature radius is equal to a diameter of the inner surface of the frame 21. With such a spherical shape, the ultraviolet light reflected by the mirror surface (concave surface) 22a is focused in a vicinity of a facing surface, so that the amount of the light leaking to the outside of the frame 21 can be further prevented.

The shape of the mirror surface (concave surface) 22a may be a paraboloidal surface, that is, a paraboloid structure centered on an optical axis of an incident ultraviolet light such that the ultraviolet light emitted by spreading from the ultraviolet ray light source 20 is reflected in parallel by the mirror surface (concave surface) 22a in a cross section parallel to the thickness direction of the frame 21. With such a shape, when the ultraviolet light reflected by the mirror surface (concave surface) 22a is next reflected by the facing mirror surface (concave surface) 22a, the ultraviolet light is focused in the vicinity of the facing surface, so that the amount of the light leaking to the outside of the frame 21 can be further prevented.

FIG. 3 is a cross-sectional view taken along a cross section perpendicular to the thickness direction of the cylindrical air sterilizer illustrated in FIG. 1. As illustrated in FIG. 3, an odd number of the ultraviolet ray light sources 20 are disposed uniformly on the inner surface side of the frame 21 along the circumferential direction. In particular, the ultraviolet ray light sources 20 are not disposed on the facing surfaces of the ultraviolet ray light sources 20. If the ultraviolet ray light source 20 is disposed on the facing surface of the ultraviolet ray light source 20, the ultraviolet light may be absorbed and the reflectance may be decreased. By causing the ultraviolet ray light source 20 to face the mirror surface 22a across a center of the frame 21, a decrease in the reflectance can be prevented. In this way, by uniformly disposing an odd number of three or more of the ultraviolet ray light sources 20 such that the facing surfaces are mirror surfaces, the ultraviolet light density inside the frame 21 can be increased even if a light output intensity of one ultraviolet ray light source 20 is low.

### Second Embodiment

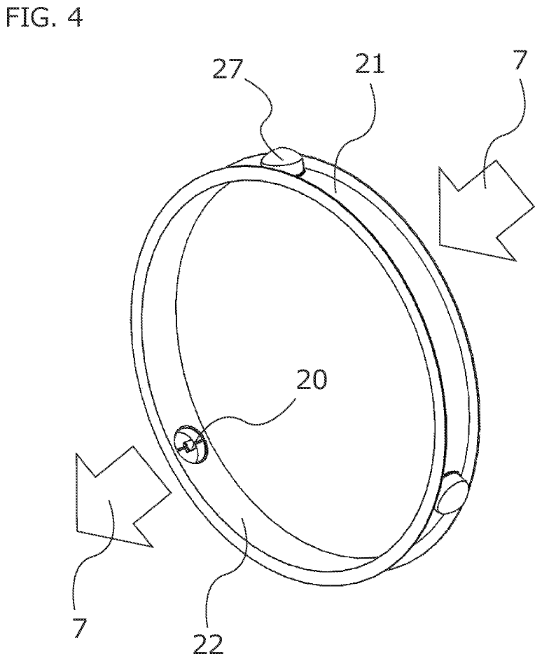

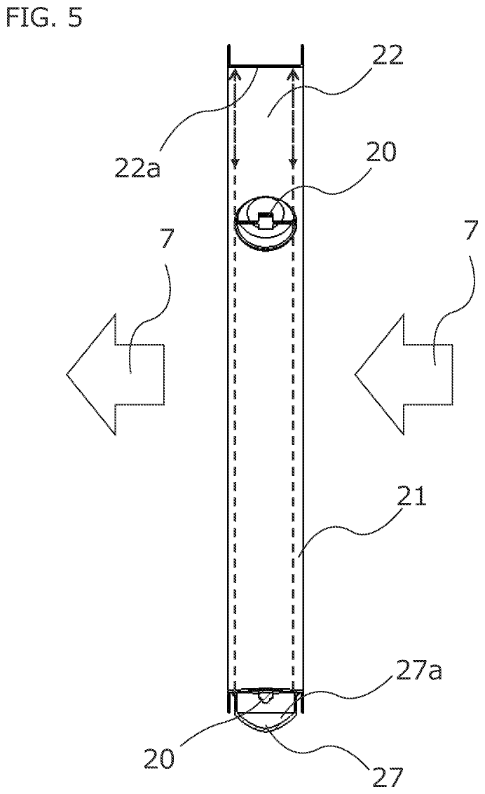

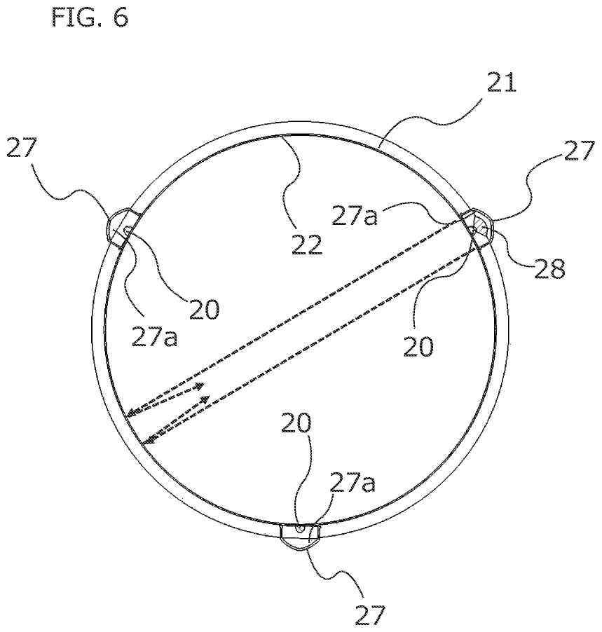

A second embodiment of the invention will be described with reference to FIGS. 4, 5, and 6. FIG. 4 is a perspective view of an air sterilizer according to the second embodiment. FIGS. 5 and 6 are views illustrating cross-sectional structures of the air sterilizer. However, the invention is not limited to these embodiments.

As illustrated in FIG. 4, in the air sterilizer of the present embodiment, the plurality of ultraviolet LEDs as the ultraviolet ray light sources 20 are mounted inside the cylindrical frame 21 along the circumferential direction. The inner surface of the frame 21 is the mirror surface portion 22, circular opening portions are provided in a part thereof, and the ultraviolet ray light sources 20 are supported at centers of the opening portions via support members. The emission surface of the ultraviolet ray light source 20 faces the outside of the frame 21, and the ultraviolet light 28 (FIG. 6) emitted from the emission surface is reflected by a mirror surface 27a of a reflection case 27 on the outside of the frame 21, and is emitted from the opening portion to the inside of the frame 21 (see dotted lines in FIGS. 5 and 6).

The mirror surface portion 22 has the mirror surface 22a that can reflect the ultraviolet light emitted from the opening portion to the inside of the frame 21 with high reflectance. The mirror surface 22a has a function of repeatedly reflecting the ultraviolet light emitted radially inward to keep the ultraviolet light inside the frame 21 of the cylindrical shape and to increase the ultraviolet light density of the cylindrical inner surface.

Air flows in the direction indicated by the arrow of the air flow 7. That is, air flows in the thickness direction (that is, the axial direction) of the cylindrical frame 21.

As illustrated in FIG. 4, the diameter of the cylindrical inner circumferential surface of the frame 21 is larger than the axial thickness of the frame 21. This is because a duct through which air flows of an air conditioning apparatus or the like includes a structure such as a heat exchanger or a filter, and the air sterilizer mounted in an empty space in the duct preferably has a thin shape with respect to the air flow.

FIG. 5 is a cross-sectional view taken along a cross section parallel to the thickness direction of the cylindrical air sterilizer illustrated in FIG. 4. As illustrated in FIG. 5, the plurality of ultraviolet ray light sources 20 are provided so as to be embedded on a center cross section in the thickness direction on the inner surface side of the frame 21. In the first embodiment, the ultraviolet ray light source 20 has the emission surface facing inward so as to emit ultraviolet light toward a center direction and inward of the frame 21, but in the present embodiment, the ultraviolet ray light source 20 has an emission surface facing outward so as to emit ultraviolet light toward the outside of the frame 21.

The reflection case 27 is disposed outside the frame 21 so as to cover the opening portion, and a surface of the reflection case 27 facing the ultraviolet ray light source 20 is the mirror surface (concave surface) 27a. In the present embodiment, the mirror surface (concave surface) 27a inside the reflection case 27 reflects light spread from the ultraviolet ray light source 20 in a shape close to parallel light (at a divergence angle (including a case where the divergence angle is 0°) or at a convergence angle smaller than the divergence angle of the ultraviolet light), and supplies the light to the inside of the frame 21. Therefore, the support member that supports the ultraviolet ray light source 20 with respect to the opening portion preferably has a thin shape such as a column.

In the present embodiment, the inner surface of the frame 21 itself is the cylindrical mirror surface portion 22, and is not spherical. However, since the frame 21 itself is cylindrical, ultraviolet light emitted from the mirror surface 27a travels as parallel light, and when the ultraviolet light is reflected by the facing mirror surface 22a, the ultraviolet light is reflected in an elliptical shape. More specifically, a cross-sectional length of the reflected light does not change in the thickness direction of the frame 21 (see the dotted line in FIG. 5), but decreases in a direction orthogonal to the thickness direction of the frame 21 (see the dotted line in FIG. 6). Therefore, the ultraviolet light density inside the frame can be made uniform.

However, the concave mirror surface 22a may be provided on the inner surface of the frame 21, and in this case, it is considered that ultraviolet light leaking to the outside of the frame 21 in the thickness direction is prevented by setting a curvature radius of the concave surface to be sufficiently larger than the diameter of the frame 21.

FIG. 6 is a cross-sectional view taken along a cross section perpendicular to the thickness direction of the cylindrical air sterilizer illustrated in FIG. 4. As illustrated in FIG. 6, the ultraviolet ray light sources 20 are uniformly disposed on the inner surface side of the frame 21 along the circumferential direction. In particular, the ultraviolet ray light source 20 is not disposed at a position where the ultraviolet ray light source 20 faces a rear surface of an emission surface of an ultraviolet ray light source 20. If the ultraviolet ray light source 20 is disposed at the facing position, the ultraviolet light emitted into the frame 21 via the reflection case 27 may be incident on and absorbed by the facing ultraviolet ray light source 20. By not disposing the ultraviolet ray light source 20 at the position where the ultraviolet ray light source 20 faces the rear surface of the emission surface of the ultraviolet ray light source 20, a decrease in the reflectance can be prevented.

In this way, by uniformly disposing the ultraviolet ray light sources 20 in an odd number of three or more such that the surfaces facing each other are mirror surfaces, the ultraviolet light density inside the frame 21 can be increased even if the light output intensity of one ultraviolet ray light source 20 is weak.

A structure of the mirror surface (concave surface) 22a of the reflection case 27 facing the ultraviolet ray light source 20 is preferably a paraboloidal surface, that is, a paraboloid structure, in order to reflect the divergent light of the ultraviolet ray light source 20 and generate parallel light as much as possible.

### Third Embodiment

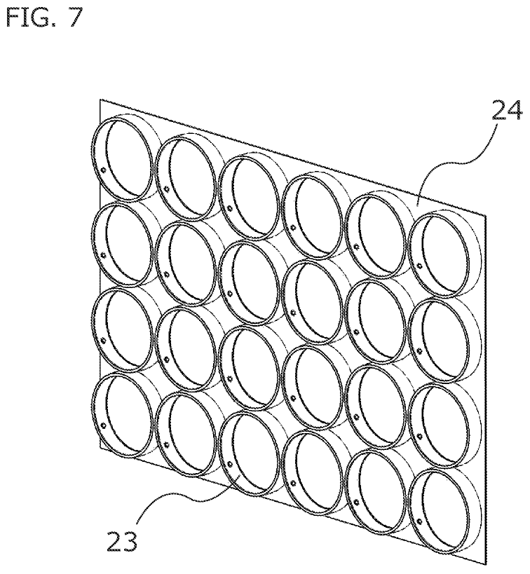

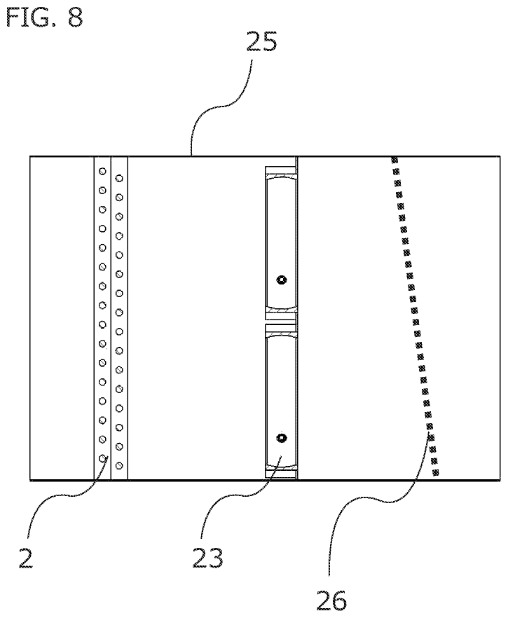

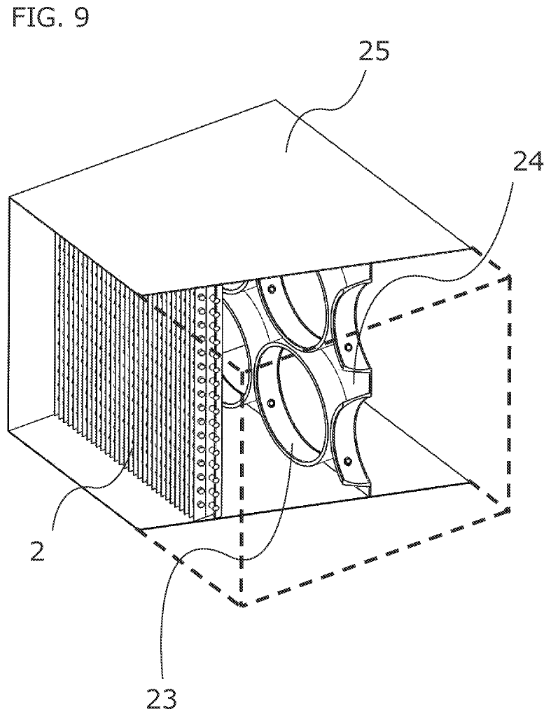

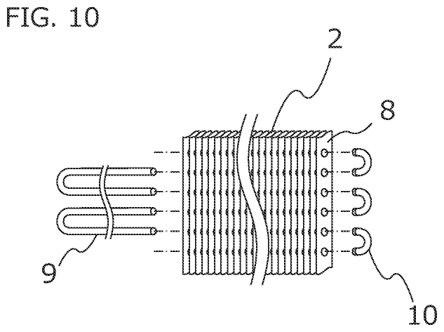

A third embodiment of the invention will be described with reference to FIGS. 7, 8, 9, and 10. FIG. 7 is a diagram illustrating a mounting structure of an air sterilizer according to the third embodiment. FIG. 8 is a cross-sectional view of a duct configuration using the air sterilizer according to the third embodiment, and FIG. 9 is a diagram illustrating a partial cut model of the duct configuration. FIG. 10 is a diagram illustrating components of a fin tube heat exchanger used in the third embodiment. However, the invention is not limited to these embodiments.

In the present embodiment, as illustrated in FIG. 7, a plurality of air sterilizers 23 illustrated in the first embodiment and the second embodiment are mounted on a frame 24. The frame 24 has an opening that does not obstruct an air flow passing through the frame 21. Accordingly, even when a flow rate at which air flows while inactivating bacteria, viruses, and the like by one air sterilizer 23 is small, the bacteria, the viruses, and the like in a large amount of flowing air can be treated at once by using the plurality of air sterilizers 23.

As illustrated in FIG. 8, the frame 24 illustrated in FIG. 7 can be mounted in an air conditioning duct 25, through which air flows, of an air conditioner or the like. The number of the air sterilizers 23 mounted on the frame 24 can be freely changed according to a cross-sectional area of the air conditioning duct 25. In particular, a structure of the present embodiment is thin and thus can be easily mounted between a heat exchanger 2 of the air conditioning duct 25 and a filter 26. The filter 26 is formed of a metal wire mesh or the like, and has a function of removing dust and the like in air flowing in the air conditioning duct 25. The heat exchanger 2 has a chlorofluorocarbon-based refrigerant, water, and the like flowing inside, and has a function of cooling or warming a temperature of passing air. In particular, when air is cooled in the heat exchanger 2, dew condensation water may be generated on a surface of the heat exchanger 2 depending on conditions.

FIG. 9 is a cut model illustrating an inside of a structure of the air conditioning duct 25 illustrated in FIG. 8. Since it is surrounded by the frame 24, all air flows in the air conditioning duct 25 pass through the air sterilizers 23.

Further, as illustrated in FIG. 10, the heat exchanger 2 is of a type called a fin tube type. As illustrated in FIG. 10, the heat exchanger 2 has a structure in which a heat transfer pipe 9 formed on a hairpin penetrates a large number of flat plate-shaped fins 8. An end portion of the heat transfer tube is connected by a contact such as a return pipe 10, and a meandering refrigerant flow path is formed by inside of the heat transfer pipe 9 and the return pipe 10 which form a closed circuit.

The air sterilizer 23 of the present embodiment has a relatively small thickness, and thus the ultraviolet light is likely to leak in the thickness direction. Since the heat exchanger 2 is a fin tube type heat exchanger including a large number of flat plate-shaped fins, bacteria, viruses, and the like on a fin surface can be inactivated by irradiation of ultraviolet light leaked from the air sterilizer 23. In particular, generation of mold which is a source of odor can be prevented by ultraviolet light irradiation.

On the other hand, when the ultraviolet light of the air sterilizer 23 leaks to a side opposite to the heat exchanger 2, the filter 26 is irradiated with the ultraviolet light. When the filter 26 is made of metal, the filter 26 is not degraded by the ultraviolet light, but rather the ultraviolet light can be expected to inactivate bacteria, viruses, and the like of dust deposited on a surface of the filter 26, decompose some of the dust and reduce the amount of the deposited dust.

Most of the ultraviolet light leaking in the axial direction of the air sterilizer 23 is absorbed by the heat exchanger 2 and the filter 26, but some of the ultraviolet light may further leak outward through gaps between the fins of the heat exchanger 2 or through gaps in the filter. Therefore, it is preferable that blower fans are disposed at both ends of the air conditioning duct 25 so as not to leak the ultraviolet light, or the air conditioning duct 25 is formed in a grille shape such that the leaked ultraviolet light does not directly go out of the air conditioning duct.

### Fourth Embodiment

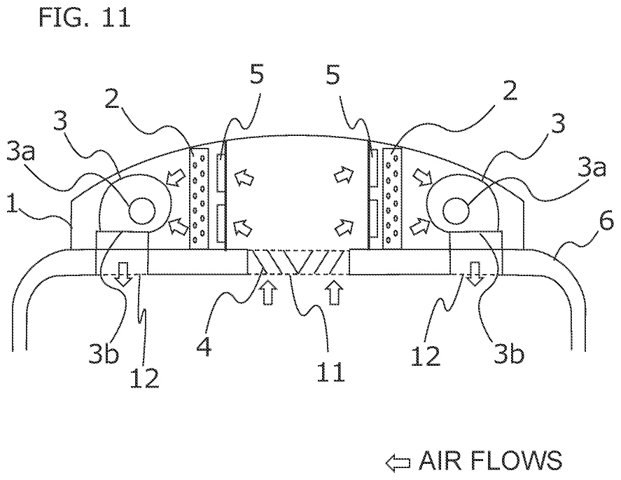

A fourth embodiment of the invention will be described with reference to FIG. 11. FIG. 11 illustrates a mounting structure of the air sterilizer according to the present embodiment on an air conditioning apparatus for a railway vehicle. In FIG. 11, arrows illustrate air flows. However, the invention is not limited to these embodiments.

As illustrated in FIG. 11, an air conditioner (air conditioning apparatus) 1 is mounted on a roof of a vehicle body 6. When the air conditioner 1 is operated, air flows generated by blowers 3 on both sides causes air in an interior of the vehicle to be introduced into an interior of the air conditioner 1 through an air inlet 11 in which a suction grill 4 is provided. Although not illustrated, the introduced air passes through a filter, passes through air sterilizers 5, is cooled or heated by the heat exchangers 2, passes through the blowers 3, and returns into the interior of the vehicle from air outlets 12. The air sterilizer 5 has the same configuration as that of the above-described embodiment.

A centrifugal fan is used as the blower 3. In general, a centrifugal fan or a sirocco fan uses a form in which air is sucked in a direction of a rotation axis thereof and air is blown out in a direction perpendicular to the rotation axis. In the present embodiment, an air suction direction of the blower 3 coincides with a longitudinal direction of the vehicle body. The suction grill 4 is formed by disposing a plurality of substantially flat plate-shaped blades in substantially the same longitudinal direction. An angle and a length of the blade in the flow direction are set such that a blade surface intersects with a straight line connecting an ultraviolet ray light source of the air sterilizer 5 and the interior of the vehicle, so that ultraviolet ray from the air sterilizer 5 does not pass therethrough. A fluorescent substance is applied to the blade surface.

The heat exchanger 2 to which dust, bacteria, viruses, and the like are likely to adhere is irradiated with ultraviolet light leaking from the air sterilizer 5, and the ultraviolet light exerts effects of embrittlement of dust and inactivation of the bacteria, the viruses, and the like. On the other hand, among the ultraviolet light leaking from the air sterilizer 5, the ultraviolet light traveling toward the side opposite to the heat exchanger 2 is emitted to the filter, an upper cover, and the suction grill 4 of the air conditioner 1.

As described above, since the suction grill 4 is disposed such that the angle and the length in the flow direction of the blade constituting it intersect the straight line connecting the ultraviolet ray light source and the interior of the vehicle, the ultraviolet light can be prevented from directly leaking into the interior of the vehicle and being emitted to passengers in the interior of the vehicle. Further, since the ultraviolet light emitted to the blade surface fluorescent substance applied to the blade surface to emit light, an operation of the ultraviolet ray light source can be checked from the interior of the vehicle, which can appeal to the passengers with a sense of security and cleanliness.

As described above, according to the present embodiment, bacteria, viruses, and the like in air passing through the air conditioner 1 can be inactivated, and the surfaces of the heat exchanger 2 and the filter can be irradiated with the ultraviolet light, effects of embrittlement of dust and disinfection of bacteria, viruses, and the like can be exerted. Further, it is possible to provide an air conditioner for a railway vehicle that can prevent leakage of the ultraviolet light into the interior of the vehicle and can safely supply air with excellent cleanliness.

### Fifth Embodiment

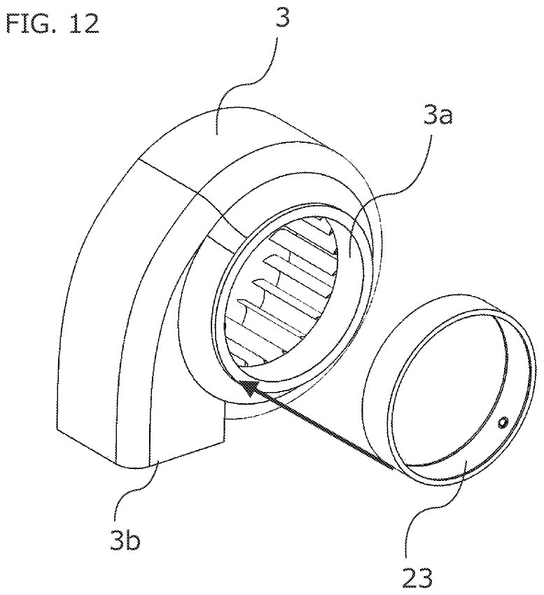

A fifth embodiment of the invention will be described with reference to FIG. 12. FIG. 12 illustrates a mounting structure of the air sterilizer. However, the invention is not limited to these embodiments.

As illustrated in the air conditioning apparatus for the railway vehicle of the fourth embodiment, a sirocco fan as illustrated as the blower 3 in FIG. 12 may be used as a blower of the air conditioning apparatus and a blower duct.

In general, a centrifugal fan or a sirocco fan uses a form in which air is sucked in the direction of the rotation axis thereof and air is blown out in the direction perpendicular to the rotation axis. In FIG. 12, a structure is such that air is sucked from a blower inlet 3a on a side surface and is blown out from a downward blower outlet 3b by a blade rotating inside.

In general, in a centrifugal fan or a sirocco fan, the blower inlet 3a has a circular shape. Therefore, the air sterilizer 23 having a diameter equal to a diameter of the blower inlet 3a is fitted and disposed in the blower inlet 3a. By adopting such a structure, air containing bacteria, viruses, and the like is inactivated by the air sterilizer 23 before being sucked into the blower 3, so that clean air can be blown out from the blower outlet 3b.

When the blade rotating inside the blower 3 is made of metal, the ultraviolet light leaking from the air sterilizer 23 is reflected and absorbed by the blade, so that the amount of light leaking from the blower outlet 3b can be greatly reduced. Therefore, in a duct air conditioner or the like, even in an environment in which a person exists ahead of the blower outlet 3b, ultraviolet rays leaking from the blower outlet 3b can be prevented as much as possible when the air sterilizer 23 is disposed at the blower inlet 3a. Since a heat exchanger or the like is disposed on a side of the blower inlet 3a, even if the air sterilizer 23 is disposed on the blower inlet 3a, no particular problem is found because the leaked ultraviolet light is blocked by the heat exchanger or the like.

### Sixth Embodiment

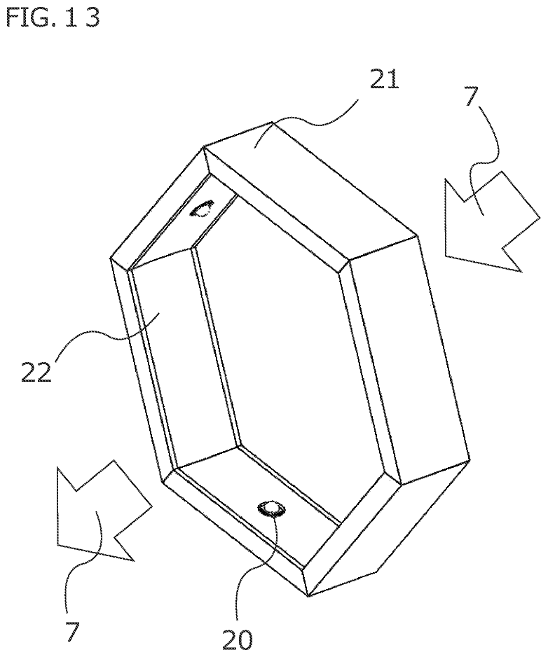

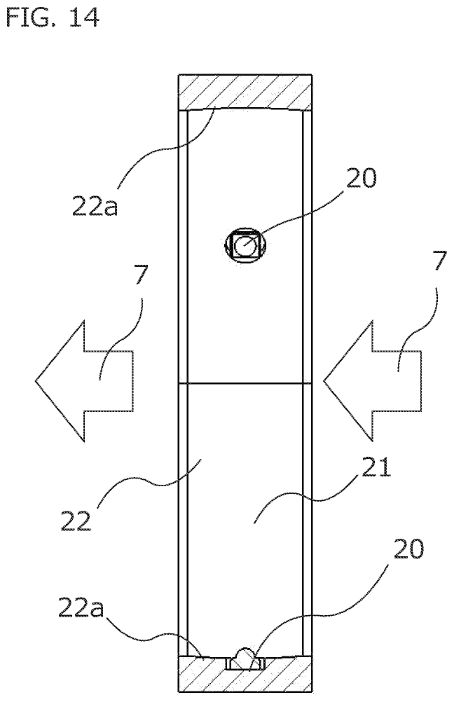

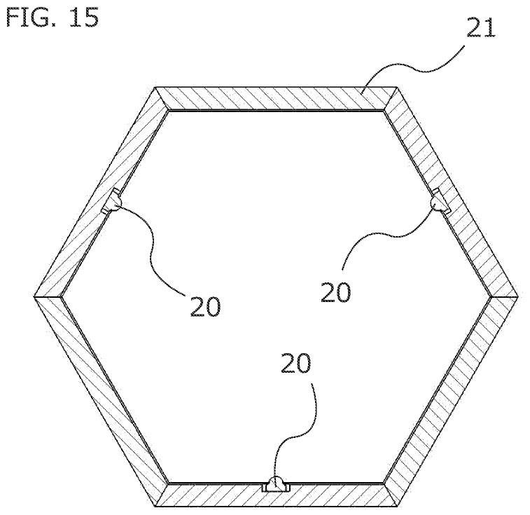

A sixth embodiment of the invention will be described with reference to FIGS. 13, 14, and 15. FIG. 13 is a perspective view of the air sterilizer according to the sixth embodiment. FIGS. 14 and 15 are views illustrating cross-sectional structures of the air sterilizer. However, the invention is not limited to these embodiments.

As illustrated in FIG. 13, in the air sterilizer of the present embodiment, the frame 21 has a hexagonal cylindrical shape. As illustrated in the present embodiment, even if the frame 21 has a polygonal cylindrical shape that is not a cylindrical shape, if the ultraviolet ray light source 20 is disposed so as not to be present on the mirror surface 22 facing the ultraviolet ray light source 20, loss of the ultraviolet light during reflection can be reduced, and the ultraviolet light density inside thereof can be easily increased. Further, by forming the frame 21 in the polygonal cylindrical shape, intervals between adjacent frames can be reduced, and the number of frames 21 disposed per unit area can be increased. A diameter of the maximum inscribed circle tangent to the inner surface of the frame 21 is larger than the axial thickness of the frame 21.

FIG. 14 is a cross-sectional view taken along a cross section parallel to the thickness direction of the air sterilizer having the hexagonal cylindrical shape illustrated in FIG. 13. As illustrated in FIG. 14, the plurality of ultraviolet ray light sources 20 are provided so as to be embedded in the center in the thickness direction on the inner surface side of the frame 21. The inner surface of the frame 21 is the mirror surface portion 22, and similarly to the first embodiment, the mirror surface 22a having the curved surface shape which is the concave surface bulging outward is provided. The ultraviolet light emitted from the ultraviolet ray light source 20 travels while spreading outward in the thickness direction of the frame 21, and is reflected inward in the thickness direction by the facing mirror surface (concave surface) 22a, so that the amount of light leaking to the outside of the frame 21 can be prevented.

As in the first embodiment, the shape of the mirror surface (concave surface) 22a may be an arc whose curvature radius is equal to a distance between the facing surfaces of the inner surface of the frame 21. Further, the shape of the mirror surface (concave surface) 22a may be a paraboloidal surface, that is, a paraboloid structure in which the light is reflected in parallel by the mirror surface (concave surface) 22a.

FIG. 15 is a cross-sectional view taken along a cross section perpendicular to the thickness direction of the air sterilizer having the hexagonal cylindrical shape illustrated in FIG. 13. As illustrated in FIG. 15, the ultraviolet ray light sources 20 are uniformly disposed on the inner surface side of the frame 21. In particular, the ultraviolet ray light sources 20 are not disposed on the facing surfaces of the ultraviolet ray light sources 20. If the ultraviolet ray light source 20 is disposed at the facing position, the ultraviolet light emitted from the ultraviolet ray light source 20 may be incident on and absorbed by the facing ultraviolet ray light source 20, and the reflectance may be decreased. Since the ultraviolet ray light source 20 is not disposed on the facing surface of the ultraviolet ray light source 20, a decrease in the reflectance can be prevented.

Therefore, by uniformly disposing the plurality of the ultraviolet ray light sources 20 such that surfaces facing to the ultraviolet ray light sources 20 are mirror surfaces, the ultraviolet light density inside the frame 21 can be increased even when the light output intensity of one ultraviolet ray light source 20 is weak. Therefore, in particular in a case of forming the air sterilizer in the polygonal shape as in the present embodiment, it is desirable to form the air sterilizer in a regular polygonal cylindrical shape having two times the number of the ultraviolet ray light sources 20, such as a hexagonal cylindrical shape when the number of the ultraviolet ray light sources 20 is three, and an octagonal cylindrical shape when the number of the ultraviolet ray light sources 20 is four.

Alternatively, in an embodiment having the frame 21 in which the mirror surface 22a is a concave curved surface, a pair of mirror surfaces having gently inclined circular (corner) conical inner circumferential surfaces may be formed, and the circular (corner) conical mirror surfaces may be combined so as to face each other to form the frame 21. The ultraviolet light emitted from the ultraviolet ray light source 20 at a predetermined divergence angle is reflected by one circular (corner) conical mirror surface and directed to a center side of the frame 21, and is reflected by the other circular (angular) conical mirror surface and similarly directed to the center side of the frame 21, so that leakage of the ultraviolet light to the outside of the frame 21 can be prevented.

According to the invention, the ultraviolet light density inside the frame can be increased by emitting the ultraviolet light using an ultraviolet LED having a relatively long life and low cost and repeatedly reflecting the ultraviolet light inside the frame. Further, since the leakage of light in the thickness direction of the frame can be prevented by the reflection of the concave mirror surface, emission of the ultraviolet light to the outside of the frame can be relatively prevented even when the thickness of the frame is reduced. With such a configuration, it is possible to realize an air sterilizer that can be mounted even in a narrow air conditioning duct, which is sandwiched between a heat exchanger and a filter, while reducing a blowing resistance.

### REFERENCE SIGNS LIST

1 air conditioner
2 heat exchanger
3 blower
3a blower inlet
3b blower outlet
4 suction grill
5 air sterilizer
6 vehicle body
7 air flow
8 fin
9 heat transfer pipe
10 return pipe
11 air inlet
20 ultraviolet ray light source
21 frame
22 mirror surface portion
22a mirror surface
23 air sterilizer
24 frame
25 air conditioning duct
26 filter
27 reflection case
27a mirror surface (concave surface)

1. An air sterilizer comprising:
a frame of which air passes through an inside;
an ultraviolet ray light source configured to emit ultraviolet light having a predetermined divergence angle; and
a mirror surface configured to reflect the ultraviolet light and emit the ultraviolet light toward the air passing through the inside of the frame, wherein
a thickness of the frame in a direction in which the air passes is smaller than a diameter of a circle tangent to an inner circumference of the frame, and
when the ultraviolet light emitted from the ultraviolet ray light source is reflected by the mirror surface, the reflected light has a divergence angle or a convergence angle smaller than the divergence angle at least in a thickness direction of the frame.

2. The air sterilizer according to claim 1, wherein
an optical axis of the ultraviolet ray light source intersects an axis of the frame.

3. The air sterilizer according to claim 1, wherein
an inner surface of the frame is the mirror surface.

4. The air sterilizer according to claim 1, wherein
an opening portion is formed in the frame, and the ultraviolet ray light source is disposed in the opening portion such that an emission surface of the ultraviolet light is directed to an outside of the frame, and the mirror surface is disposed to face the emission surface.

5. The air sterilizer according to claim 1, wherein
the mirror surface includes a spherical surface or a paraboloidal surface.

6. The air sterilizer according to claim 1, wherein
a plurality of the ultraviolet ray light sources are disposed at equal intervals along a circumferential direction of the frame.

7. The air sterilizer according to claim 6, wherein
the frame has a cylindrical shape or a polygonal cylindrical shape, and the ultraviolet ray light source faces the mirror surface across a center of the frame.

8. The air sterilizer according to claim 1, wherein
the ultraviolet ray light source is an ultraviolet ray light emitting diode that emits ultraviolet rays having a wavelength of 200 nm to 300 nm.

9. An air conditioning apparatus comprising:
the air sterilizer according to claim 1; and
a heat exchanger or a filter in an air conditioning duct, wherein
the air sterilizer is disposed on an upstream side of the heat exchanger or on a downstream side of the filter.

10. The air conditioning apparatus according to claim 9, wherein
a plurality of the air sterilizers are disposed on a frame provided in the air conditioning duct in parallel with respect to an air flow.

11. The air conditioning apparatus according to claim 9, wherein
the heat exchanger or the filter is irradiated with some of ultraviolet rays emitted from the air sterilizer.

12. The air conditioning apparatus according to claim 9 further comprising:
a blower configured to generate an air flow, wherein
the frame of the air sterilizer is provided at a blower inlet of the blower.
